(19) United States
(12) Patent Application Publication
(10) Pub. No.: US 2013/0035587 A1
(43) Pub. Date: Feb. 7, 2013

(54) Radiotherapy Apparatus

(71) Applicant: ELEKTA AB (PUBL), Stockholm (SE)

(72) Inventors: Jan Lagendijk, Utrecht (NL); David Jaffray, Etobicoke (CA)

(73) Assignee: ELEKTA AB (PUBL), Stockholm (SE)

(21) Appl. No.: 13/647,640

(22) Filed: Oct. 9, 2012

Related U.S. Application Data
(63) Continuation of application No. PCT/EP2010/002324, filed on Apr. 15, 2010.

(51) Int. Cl. A61N 5/10; A61B 6/00; A61B 5/055

(52) U.S. Cl. 600/411

(57) ABSTRACT

A radiotherapy apparatus is described which includes a patient support, a magnetic resonance imaging (MRI) apparatus for obtaining imaging data of a patient positioned on the patient support, and multiple linear accelerators, each aligned in a plane transverse to the patient support and arranged to provide a therapeutic beam of x-ray radiation to the patient.

This Application is a continuation of Patent Cooperation Treat Patent Application PCT/EP2010/002324 filed Apr. 15, 2010, which is incorporated herein by reference.

### TECHNICAL FIELD

The present invention relates to radiotherapy systems, and particularly to radiotherapy systems comprising integrated magnetic resonance imaging apparatus.

### BACKGROUND ART

Current designs for combining magnetic resonance imaging (MRI) with radiotherapy apparatus in the form of a linear accelerator (linacs) involve arranging the magnetic field coils to allow a linac to be placed in such a way as to minimize the effect of the magnetic field on its operation, and allow the treatment beam to penetrate to the patient's target region with minimal attenuation. Such a design is shown in FIG. 1.

In current designs the radiation beam has to pass through the MRI magnet and RF coils, thereby attenuating the beam. For this reason, the linac also needs to be placed at an extended distance from the patient so as to allow adequate clearance. Both of these factors have the effect of lowering the maximum achievable dose rate, which in turn increases the treatment time.

Existing treatment techniques include intensity-modulated radiation therapy (IMRT) and volumetric-modulated arc therapy (VMAT), which (for complex dose distributions) require irradiation from multiple directions or delivery of multiple arcs of radiation. Whilst these techniques allow for the delivery of complex and accurate dose distributions, they also undesirably extend the time to deliver treatment.

### SUMMARY

The present invention therefore provides, in one embodiment, a radiotherapy apparatus, comprising a patient support, a magnetic resonance imaging (MRI) apparatus, for obtaining imaging data of a patient positioned on the patient support; and a plurality of linear accelerators, each linear accelerator being configured to provide a therapeutic beam of x-ray radiation to the patient.

In conventional radiotherapy systems, the overall size of the system is dominated by the size of the linear accelerator. It is desirable that the overall size of the machine is kept to a minimum, to minimize cost and for convenience of installation. This has in the past led to a situation in which radiotherapy systems comprise only a single therapeutic linear accelerator.

In an MRI-linac combination, however, the provision of the magnetic coils forces the overall size of the machine to be much larger, leading to no additional penalty in terms of machine size from including more linear accelerators. The dominant factor in the size of the system is the MRI apparatus. Therefore, to overcome the limitations imposed by a lower maximum dose rate, embodiments of the invention describe an MRI-linac combination comprising one or more additional therapeutic linacs.

Further, in an existing linac-based radiotherapeutic apparatus, if a higher dose rate is required then this can be achieved straightforwardly by making appropriate changes to the single linac. In a combined MRI/linac device, there are additional constraints imposed by the need to package the linac so that it will remain compatible with the design of the MRI coils. This may limit the scope for making substantial changes to the linac. Meanwhile, there is adequate space around the MRI coils for two, three, four or more linacs which may be distributed around the circumference in order to provide a corresponding multiplication of the available dose rate without affecting the size or compatibility of the individual linacs.

The linear accelerators provide a therapeutic beam of x-ray radiation by emitting a beam having an energy level sufficient to have a potentially therapeutic effect. This can be contrasted with a diagnostic beam, used in conjunction with an imaging device to create a useful diagnostic image of the patient having good contrast. Typically, the energies of therapeutic and diagnostic beams differ by several orders of magnitude, with therapeutic beams in the MeV range and diagnostic beams in the keV range.

### BRIEF DESCRIPTION OF THE DRAWINGS

FIG. 1 shows a conventional radiotherapy system comprising an MRI apparatus.

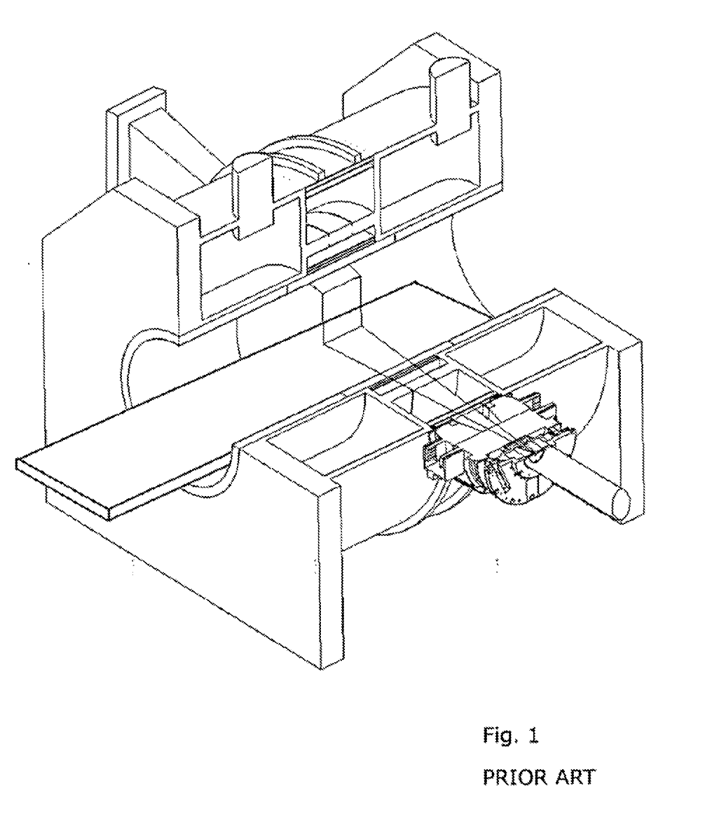

FIG. 2 is a cross-section through a radiotherapy-MRI combination according to embodiments of the present invention.

FIG. 3 is a schematic diagram of aspects of the radiotherapy system according to embodiments of the present invention.

FIG. 4 is a beam's eye view of the target region and imaging planes according to embodiments of the present invention.

FIG. 5 shows a view of the target region and the imaging lines according to other embodiments of the present invention.

### DETAILED DESCRIPTION

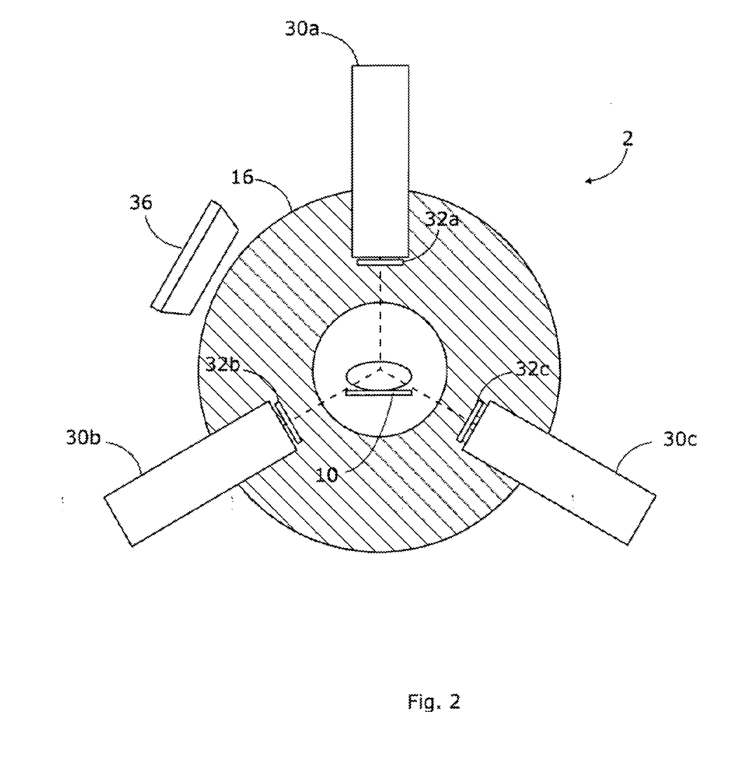

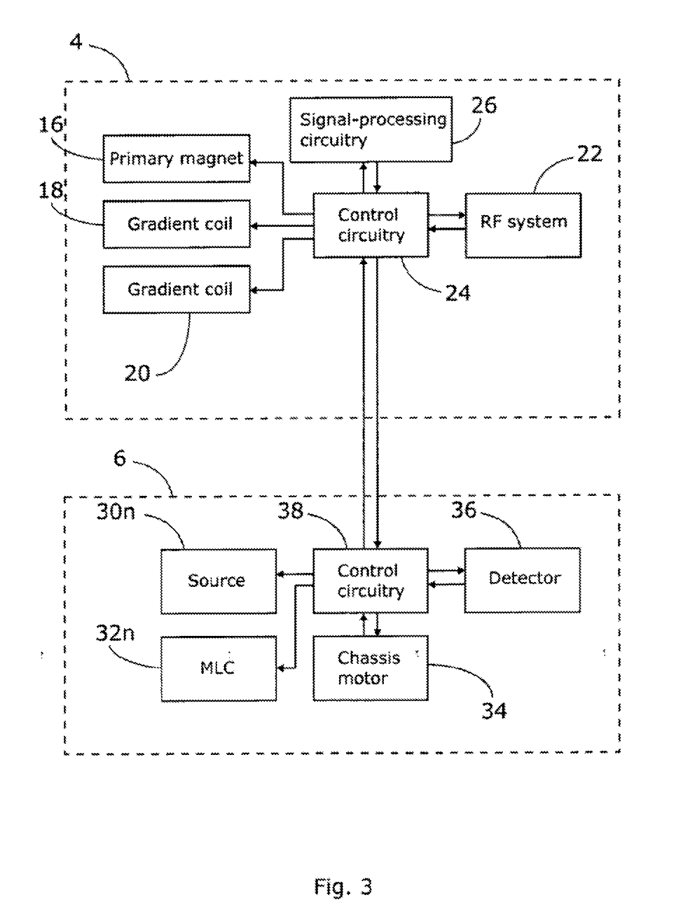

FIG. 2 is a cross-section view of a system according to embodiments of the present invention, comprising a radiotherapy apparatus and a magnetic resonance imaging (MRI) apparatus. The radiotherapy apparatus 6 and MRI apparatus 4 are shown schematically in FIG. 3.

The system includes a couch 10, for supporting a patient in the apparatus. The couch 10 is movable along a horizontal, translation axis (into the page of FIG. 2), such that a patient resting on the couch is moved into the radiotherapy and MRI apparatus. In one embodiment, the couch 10 is rotatable around a central vertical axis of rotation, transverse to the translation axis, although this is not illustrated. The couch 10 may form a cantilever section that projects away from a support structure (not illustrated). In one embodiment, the couch 10 is moved along the translation axis relative to the support structure in order to form the cantilever section, i.e. the cantilever section increases in length as the couch is moved and the lift remains stationary. In another embodiment, both the support structure and the couch 10 move along the translation axis, such that the cantilever section remains substantially constant in length, as described in our U.S. patent application Ser. No. 11/827,320 filed on 11 Jul. 2007; which is incorporated herein by reference.

As mentioned above, the system 2 comprises an MRI apparatus 4, for producing near real-time imaging of a patient positioned on the couch 10. The MRI apparatus includes a primary magnet 16 which acts to generate the so-called “primary” magnetic field for magnetic resonance imaging. That is, the magnetic field lines generated by operation of the magnet 16 run substantially parallel to the central translation axis. The primary magnet 16 consists of one or more coils with an axis that runs parallel to the translation axis of the couch. The one or more coils may be a single coil or a plurality of coaxial coils of different diameter, as illustrated. In one embodiment, the one or more coils in the primary magnet 16 are spaced such that a central window of the magnet 16 is free of coils. In other embodiments, the coils in the magnet 16 may simply be thin enough that they are substantially transparent to radiation of the wavelength generated by the radiotherapy apparatus. The magnet 16 may further comprise one or more active shielding coils, which generates a magnetic field outside the magnet 16 of approximately equal magnitude and opposite polarity to the external primary magnetic field. The more sensitive parts of the system 2, such as the accelerator, are positioned in this region outside the magnet 16 where the magnetic field is cancelled, at least to a first order. The MRI apparatus 4 further comprises two gradient coils 18, 20, which generate the so-called “gradient” magnetic field that is superposed on the primary magnetic field. These coils 18, 20 generate a gradient in the resultant magnetic field that allows spatial encoding of the protons so that their position can be determined The gradient coils 18, 20 are positioned around a common central axis with the primary magnet 16, and may be displaced from one another along that central axis. This displacement creates a gap, or window, between the two coils 18, 20. In an embodiment where the primary magnet 16 also comprises a central window between coils, the two windows are aligned with one another.

An RF system 22 causes the protons to alter their alignment relative to the magnetic field. When the RF electromagnetic field is turned off the protons return to the original magnetization alignment. These alignment changes create a signal which can be detected by scanning. The RF system 22 may include a single coil that both transmits the radio signals and receives the reflected signals, dedicated transmitting and receiving coils, or multi-element phased array coils, for example. Control circuitry 24 controls the operation of the various coils 16, 18, 20 and the RF system 22, and signal-processing circuitry 26 receives the output of the RF system, generating therefrom images of the patient supported by the couch 10.

As mentioned above, the system 2 further comprises a radiotherapy apparatus 6 which delivers doses of radiation to a patient supported by the couch 10.

The radiotherapy system according to embodiments of the present invention comprises a plurality of linear accelerators 30n directed towards the patient, each linear accelerator configured to provide a therapeutic dose of radiation to the patient. In the illustrated embodiment, three linear accelerators 30a, 30b, 30c are provided; however, any number greater than or equal to two is contemplated such as two, three, four or more. The linear accelerators are (in this embodiment) spaced regularly around the couch 10, i.e. with a regular angle between adjacent linear accelerators. This may simplify the arrangements for driving the linacs and for controlling them. Alternatively, they may be spaced in an alternative irregular arrangement or independently driven so that they can take up any non-co-incident position.

Each accelerator operates by accelerating a beam of electrons into a target (e.g. a heavy metal such as tungsten). The electrons decelerate rapidly, and this produces x-ray radiation having a characteristic spectrum of energies. Each accelerator 30n is associated with a respective multi-leaf collimator 32n, which shapes and directs the radiation generated by the accelerator into a beam of appropriate shape for the target region in the patient (e.g. a tumour). In the illustrated embodiment, one linear accelerator 30c is designed to operate at a different, lower energy, for example MV levels for the purpose of MVCT and external contour measurements. An imager 36 is positioned substantially opposite this linear accelerator in order to detect the imaging radiation. Alternatively, a flexible imager (not illustrated) may be positioned in the gap of the gradient coil, inside the magnet. Such a solution will give the entrance and exit beam at once, without the shadow of the exit beam passing through the magnet structures. Each linear accelerator 30n shares a common power source 31, to minimize cost and the number of additional components in the machine.

The accelerators 30n, multi-leaf collimators 32n and imager 36 are mounted on a chassis (not illustrated), which is continuously rotatable around the couch 10 when it is inserted into the treatment area, powered by one or more chassis motors 34. The radiotherapy apparatus 6 further comprises control circuitry 38, which may be integrated within the system 2 shown in FIG. 2 or remote from it, and controls the accelerators 30n, the MLCs 32n, the chassis motor 34 and the imager 36.

The linear accelerators 30s are positioned to emit beams of radiation through the window defined by the two gradient coils 18, 20, and also through the window defined in the primary magnet 16. The radiation beams may be cone beams or fan beams, for example.

In operation, a patient is placed on the couch 10 and the couch is inserted into the treatment area defined by the magnetic coils 16, 18 and the chassis.

The control circuitry 38 controls the linear accelerators 30n, the MLCs 32n and the chassis motor 34 to deliver radiation to the patient through the window between the coils 16, 18. The chassis motor 34 is controlled such that the chassis rotates about the patient, meaning the radiation can be delivered from different directions. The MLCs 32n have a plurality of elongate leaves oriented orthogonal to the beam axis. The leaves of the MLCs 32n are controlled to take different positions blocking or allowing through some or all of the radiation beam, thereby altering the shapes of the beams as they will reach the patient. Simultaneously with rotation of the chassis about the patient, the couch 10 may be moved along a translation axis into or out of the treatment area (i.e. parallel to the axis of rotation of the chassis). With this simultaneous motion a helical radiation delivery pattern is achieved, known to produce high quality dose distributions.

The advantages of this are manifold. Dose rate is increased without having to change the design of the linear accelerator. This will decrease the treatment time. Effectively, multiple arcs or multiple beam directions can be delivered simultaneously. This can decrease the treatment time, decrease the speed at which the chassis needs to rotate (thus reducing the mechanical demands on the machine), or a combination of both Alternatively, the equivalent of a full treatment arc can be accomplished with a one-third rotation of the linear accelerator chassis, making it possible to reduce the treatment time taken to deliver a single arc by as much as two thirds (for a system having three linear accelerators).

The system has built-in redundancy. If one linear accelerator develops a fault, it can be deactivated and the system continue to operate with the remaining linear accelerators.

Each linear accelerator can be aimed at a different target (should more than one target be present), allowing simultaneous treatment of different targets that might not have been within the range of a single linear accelerator's field of view. This would also reduce the movement requirements on each MLC, compared to a single MLC collimating for multiple targets.

The MRI apparatus 4, and specifically the signal-processing circuitry 26, delivers real-time (or in practice near real-time) imaging data of the patient to the control circuitry 38. This information allows the control circuitry to adapt the operation of the MLCs 32n, for example, such that the radiation delivered to the patient accurately tracks the motion of the target region, for example due to breathing.

As mentioned above, conventionally, an MRI apparatus would be used to obtain a full three-dimensional image of the patient. However, this can take a relatively long time, increasing the delay between acquisition of the imaging data, and provision of the data to the multi-leaf collimators. According to embodiments of the present invention, the MRI apparatus 4 is configured to obtain imaging data comprising two-dimensional slices or one-dimensional line profiles through the target region or regions of a patient, as described in detail below. Such imaging data may then be provided to the control circuitry 38 to allow the radiation beams to be shaped and directed to the target region (or target regions) as appropriate.

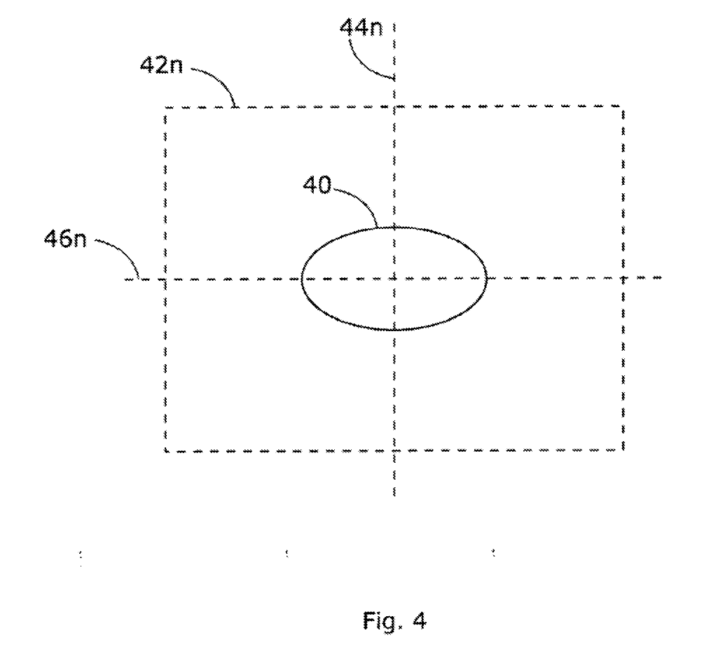

FIG. 4 shows a beam's eye view of a target area 40 in a patient (e.g. a tumour), and the imaging planes according to embodiments of the invention for one of the plurality of linear accelerators 30n. These imaging planes are obtained for each linear accelerator 30n, with respective imaging data informing the positions of respective multi-leaf collimators 32n.

The imaging data obtained by the MRI apparatus 4 comprises at least a two-dimensional slice image of a plane 42 through the target region 40, oriented orthogonally to the axis of the beam (which is into the paper in the illustrated example). The imaging data may comprise a plurality of slice images oriented in this direction or, in one embodiment, only a single two-dimensional slice image oriented in this direction. This plane 42 allows the position of the target 40 to be visualized and the position or shape of the beam adjusted accordingly by appropriate positioning of the leaves of the MLC 32n. It can be seen that in this embodiment the position of the target will only be known in two dimensions, due to the single slice, but it is also true that the adjustment of the radiation beam position (i.e. by the leaves of the collimator 32n) is also only in those same two dimensions. In this way, an image of the target region 40 can be obtained rapidly, and supplied to the collimator 32 for appropriate shaping of the radiation beam before the target moves a significant distance.

According to further embodiments, however, the imaging data obtained by the MRI 4 further comprises one or two two-dimensional slice images of planes oriented orthogonally to the first plane 42. For example, FIG. 4 shows a second plane 44 that is orthogonal to the first plane 42, and a third plane 46 that is orthogonal to both first and second planes 42, 44. These three planes may be used to more accurately define the position and shape of the target region 40. All three slice images may still be obtained in a relatively short period of time compared to a full three-dimensional image. As the beams move around the patient, the angle at which the slices 42 are taken is also altered so as to maintain their substantially orthogonal relationship with their respective radiation beam. The angles of the other two planes 44, 46 may also be altered so as to maintain their substantially orthogonal relationship with the respective first plane 42.

In an alternative aspect of the invention, the angle at which the two-dimensional slice image is taken may not maintain a substantially orthogonal relationship with the radiation beam as the beam rotates around the patient, but rather be in a substantially fixed orientation relative to the patient. In this aspect, only a single slice image is obtained in any one direction, i.e. slice image 42 is the only image oriented in that particular direction. Further slice images may be obtained in further directions, however. For example, mutually perpendicular slice images 44 and 46 may be obtained, provided these are the only ones oriented in their respective directions. In this embodiment, only a single set of imaging data is obtained and this imaging data informs the positioning of each multi-leaf collimator 32n.

This aspect of the invention has the advantage that imaging data can be obtained even more rapidly and so the delay between any target motion and the corresponding adjustment of the beam position is reduced still further. Less computational complexity is involved in obtaining slice images with fixed orientations.

The thicknesses of any of the slices discussed above may be adjusted by varying the strength of the gradient field to optimise the signal to noise ratio and also the amount of the target that is included in the particular slice image. This can be used to optimise the tracking performance.

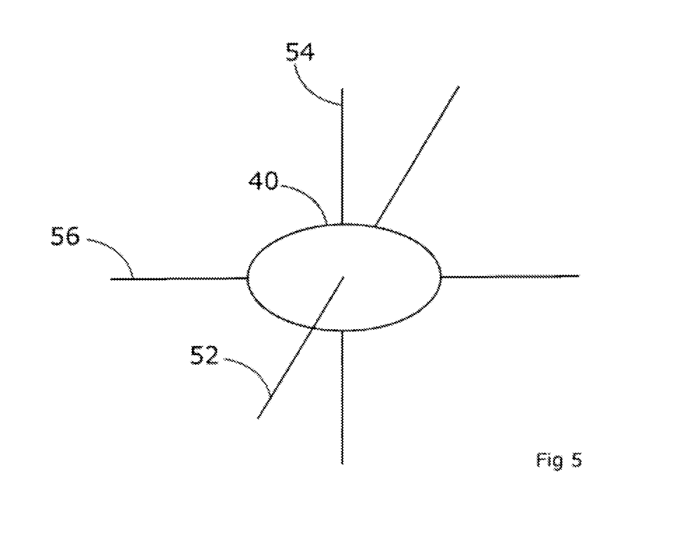

In a further alternative aspect of the invention, the imaging data comprises two or more non-parallel one-dimensional line profiles taken through the target region, such that the profiles contain boundary points between anatomical features (e.g. healthy and cancerous tissue). FIG. 5 shows imaging data according to this aspect.

The imaging data obtained by the MRI apparatus 4 in this aspect comprises at least a first one-dimensional line profile 52 through the target region 40 and a second one-dimensional line profile through the target region in a direction non-parallel to the first. In FIG. 4, three one-dimensional profiles 52, 54, 56 are illustrated, each perpendicular to the others. However, this aspect of the invention is not limited to orthogonal one-dimensional profiles.

At least one of the line profiles may be oriented substantially orthogonally to the axis of the radiation beam, with the orientation of the line profiles rotating as the radiation beam rotates around the patient to maintain that orthogonal relationship. Thus, in this embodiment, a separate set of imaging data is obtained for each radiation beam, and respective imaging data informs the positioning of respective multi-leaf collimators 32n.

In a further embodiment, the location of the profiles may change as the target region 40 moves, with a single set of imaging data informing the positioning of all multi-leaf collimators 32n.

In one embodiment, as shown in the arrangement in FIG. 5, profile 52 may be termed the “principal axis” and fixed in one particular location and one particular orientation through the target region 40. For example, the fixed direction may be the principle direction of motion of the target region, up and down the longitudinal axis of the patient due to breathing; in that case the principle axis is chosen parallel to the longitudinal axis of the patient. However, any direction of motion may be chosen in practice.

The boundary points of such a line profile 52 therefore show the movement of the target region along the principal axis. The other line profiles 54, 56 are chosen in directions that are non-parallel to the first line profile 52 and to each other (e.g. perpendicular). The orientations of the line profiles 54, 56 relative to the first line profile 52 do not change. However, the positions of the line profiles 54, 56 are not fixed and move with the movement of the target region 40 along the principle axis. Thus the first line profile 52 measures the motion of the target region in a particular direction, and the two other line profiles 54, 56 are adjusted to track with that motion.

Thus, the target region may be imaged very simply and efficiently in three dimensions using just three one-dimensional line profiles. Such a method achieves excellent temporal resolution, increasing the accuracy with which the radiation beam may be directed.

The present invention therefore provides a radiotherapy system having a plurality of therapeutic linear accelerators and an MRI imaging apparatus. In addition, various imaging techniques are disclosed that reduce the time to acquire imaging data of a target region of a patient, thereby enabling the linear accelerators to closely follow movement of the target region.

Although various exemplary embodiments of the invention have been disclosed, it should be apparent to those skilled in the art that various changes and modifications can be made which will achieve some of the advantages of the invention without departing from the true scope of the invention.

What is claimed is:

1. A radiotherapy apparatus, comprising:
a patient support;
a magnetic resonance imaging (MRI) apparatus for obtaining imaging data of a patient positioned on the patient support; and
a plurality of linear accelerators, each aligned in a plane transverse to the patient support and arranged to provide a therapeutic beam of x-ray radiation to the patient.

2. A radiotherapy apparatus according to claim 1, wherein the plurality of linear accelerators are regularly spaced around the patient support.

3. A radiotherapy apparatus according to claim 1, further comprising a plurality of collimating apparatuses, each fitted to one of the linear accelerators so as to collimate the output thereof.

4. A radiotherapy apparatus according to claim 3, wherein each collimating apparatus is separately controllable, such that radiation from different linear accelerators may be simultaneously directed towards different target regions in the patient.

5. A radiotherapy apparatus according to claim 1, wherein each of said plurality of linear accelerators are rotatable about a longitudinal axis of the patient.

6. A radiotherapy apparatus according to claim 5, wherein each of said plurality of linear accelerators emits a beam that coincides with the longitudinal axis.

7. A radiotherapy apparatus according to claim 6, wherein the beams emitted by each of said plurality of linear accelerators coincide with the longitudinal axis at substantially the same point.

8. A radiotherapy apparatus according to claim 5, wherein the imaging data comprises at least a respective plurality of first two-dimensional slice images, each first slice image including a target region of the patient and being oriented substantially orthogonal to the respective radiation beam, and wherein each collimating apparatus is controllable in dependence on its respective first slice image.

9. A radiotherapy apparatus according to claim 5, wherein the imaging data comprises at least first and second non-parallel one-dimensional line profiles, the first and second line profiles extending through the target region, and indicating boundary points of the target region in two non-parallel directions, and wherein each collimating apparatus is controllable in dependence on the imaging data.

10. A radiotherapy apparatus according to claim 5, wherein the imaging data comprises at least one two-dimensional slice image, the at least one two-dimensional slice image comprising a single two-dimensional slice image including the target region and oriented in any one direction, and wherein each collimating apparatus is controllable in dependence on the imaging data.

11. A radiotherapy apparatus according to claim 1, wherein said plurality of, linear accelerators, are rotatable about, a longitudinal axis of the patient, and wherein the or each respective plane of said imaging data is altered so as to maintain its or their substantially orthogonal relationship with the respective radiation beam.

12. A radiotherapy apparatus according to claim 1, further comprising:
a further linear accelerator, configured to provide a beam of radiation at an energy suitable for imaging; and
an imager, positioned substantially opposite the further linear accelerator, configured to detect said beam of radiation and to provide imaging data of the patient.

13. A radiotherapy apparatus according to claim 1, wherein the MRI apparatus comprises at least a first magnetic coil and a second magnetic coil, the first and second magnetic coils having a common central axis parallel to a longitudinal axis of the patient support, and being displaced from one another along the central axis to form a gap therebetween, wherein the plurality of linear accelerators are arranged to provide a therapeutic dose of radiation through said gap.
